(19) United States
(12) Patent Application Publication
(10) Pub. No.: US 2022/0054072 A1
(43) Pub. Date: Feb. 24, 2022

(54) Brain Activation Matching

(71) Applicants: Robert William Kocher, McLean, VA (US); Loran Dean Ambs, Williamsburg, VA (US); Marita Mariela Rance, Arlington, VA (US); Andrew Zachary Wisti, Arlington, VA (US)

(72) Inventors: Robert William Kocher, McLean, VA (US); Loran Dean Ambs, Williamsburg, VA (US); Marita Mariela Rance, Arlington, VA (US); Andrew Zachary Wisti, Arlington, VA (US)

(21) Appl. No.: 17/479,207

(22) Filed: Sep. 20, 2021

Related U.S. Application Data
(63) Continuation-in-part of application No. 16/991,292, filed on Aug. 12, 2020, now Pat. No. 11,123,002.

(51) Int. Cl. A61B 5/378; A61B 5/00; G16H 20/00

(52) U.S. Cl. CPC A61B 5/378; G16H 20/00; A61B 5/7246

(57) ABSTRACT

A system matching psychophysiological measures of individuals with other individuals or groups of individuals using data resulting from spontaneous brain activity and presentation of stimuli to include external stimuli, internal stimuli, sensory stimuli and neurocognitive task. Templates of distinguishing features and characteristics of collected psychophysiological measures are created for individuals and aggregations of psychophysiological measures for groups of individuals sharing common attributes. In one embodiment of the system, a template is generated for individuals and groups of high performing individuals representative of target vocations. Similarity of an individual's template to the template of a high-performing group implies similarity in cognitive function, psychophysical processes, and neurophysiological processes of the individual and the high-performing group. Psychophysiological measures used to identify candidates well suited to perform a particular function of interest overcomes limitations of subjective interpretation of pencil and paper tests of prior art personnel selection systems.

### CROSS-REFERENCE TO RELATED APPLICATIONS

This application incorporates by reference the following U.S. Pat. Nos. 10,772,527 and 11,123,002.

### BACKGROUND

The field of this invention relates to electronic brain monitoring techniques.

We spend a significant amount of time and money trying to determine “who am I?”, “what do I want to be?” and “what am I naturally good at.” One of the basic questions to a child or young adult is “What do you want to be when you grow up?” Their initial response may reflect something exciting as a fireman, policeman, or sports star. Others may take a more human approach of being a nurse, doctor, or veterinarian. Many times, the basis for their decision is on something they saw on TV, Internet or heard from their peers. Others are influenced by their parents' wishes or a teacher's guidance. This “who do I want to be?” question takes a more serious course when a young high school student starts to elect specialized courses to focus on college. A high school student's college selection decision will have significant impact on the rest of his/her life. Once in college, the average student changes their major more than twice. People will normally be happy doing things aligned with their natural aptitudes. One risk is that people don't find out what they want to do until years down the road.

Employers spend a lot of time and money searching for young college graduates to train to become professionals. Yet many personnel quit for something else after years of investment. A classic example is the U.S. military, which spend billions of dollars to attract skilled individuals. The military recruits for basic and advanced training and, then invests significantly more money in specialized training for military gunners, drivers, pilots, computer operations, weapon specialists, etc. Finding personnel to train for highly specialized positions such as fighter pilots, special operations personnel, and specialized physicians is an especially expensive and time-consuming process.

Implementations of this disclosure may solve the long-standing problem of identifying candidates that are well suited to perform a particular function of interest. This can be accomplished by matching the psychophysiological response of a candidate for the function-of-interest to that which is common to a population of persons skilled in that function-of-interest exposed to the same stimuli. In one implementation, the psychophysiological response may be observed by detecting brain activation resulting from visual stimuli. The system and process are capable of also presenting any sensory stimuli and observing psychophysiological and neurological responses such as brain activity, pupillary response, eye movement, heart rate, heart rate variability, respiration, electrodermal activity, and other responses well known in the art.

Technical literature is replete with examples of distinctive differences in the personality traits of particular groups of professionals (e.g., surgeons, astronauts, pilots) compared to the general population. Examples include: “How Do Astronaut Candidate Profiles Differ from Aviation Airline Pilots?;” Aviation Psychology and Applied Human Factors 2011; Vol. 1(1):38-44; “Personality as a Predictor of Professional Behavior in Dental School;” Journal of Dental Education; Vol. 69, No. 11; 1222; and “A Psychological Profile of Surgeons and Surgical Residents,” Journal of Surgical Education; Volume 67/Number 6, 359-370.

Many persons may have strong skills yet are not aware because they may not have been exposed the areas where they have strength. An example would be a young adult that never played an instrument but has an inherent ability to do well in music if exposed. The problem is how to identify hidden skills in a person that has the ability to be great in a particular profession hut is not aware of this since he was never exposed to the profession.

Administration of standardized tests such as the Myers-Briggs or similar tests measuring knowledge, personality traits, or cognitive ability requires a substantial amount of time for the candidate to read or listen to questions and record responses on paper or electronic media. Such tests can be compromised by the self-reporting biases of the candidate being tested. The candidate has an opportunity to consider the question and shape a response suited to how the candidate wishes to be perceived or thinks he/she is rather than providing the strictly objective response.

Tests based on written or spoken stimuli can be limited in their ability to probe the full spectrum of the psyche of the candidate. Conventional tests can also limit the responses to stimuli to very simplistic binary answers or multiple-choice answers recorded by pencil, paper, or electronic means. Interpretation of test results requires subjective assessments of skilled personnel. Consequently, conventional testing to predict the suitability of persons to perform particular functions has often not proven to be reliable due to the subjective nature of the assessment.

Conventional personality type indicators classify persons in a relatively small number of specific categories. For example, Myers-Briggs classifies a person in 1 of 16 categories. Thus, conventional personality type indicators may not have the fidelity necessary to capture traits that are indicative of certain subgroups of the human population, such as certain high performing personnel.

### DESCRIPTION OF THE SEVERAL VIEWS OF THE DRAWINGS

FIG. 1 illustrates a block diagram of the steps used to select the stimuli to develop the common test in conjunction with selecting one desired skill set, selecting a group of high performing individuals and a process utilizing neural networks to develop a signature or template.

FIG. 2 illustrates a block diagram of the steps taken to repeat part of the process shown in FIG. 1 selecting a second skill set but utilizing the same tests and environments.

FIG. 3 illustrates a block diagram of the steps taken to repeat the process in FIG. 2 but this is repeated N times.

FIG. 4 illustrates a block diagram selecting individuals to be tested, testing them, and comparing their response to the signatures of special skill set groups to determine potential association of the tested individual to any of the special skill groups.

FIG. 5 illustrates a block diagram of the steps taken to assess the suitability of multiple candidates for a single function, according to an implementation of this disclosure.

FIG. 6 illustrates a block diagram of the steps taken to assess the suitability of multiple candidates for multiple functions, according to an implementation of this disclosure.

FIG. 7 illustrates instrumentation employed in the essential embodiment of the system working on psychophysiological response to external stimuli, according to an implementation of this disclosure.

FIG. 8 illustrates sensor locations for 30 sensors, according to an implementation of this disclosure.

FIG. 9 illustrates instrumentation employed to produce multiple sensor inputs and multiple psychophysiological sensor types used to record psychophysiological response data sequences to external stimuli, according to an implementation of this disclosure.

FIG. 10 shows an example computing device according to an implementation of this disclosure.

FIG. 11 shows an example network arrangement according to an implementation of this disclosure.

FIG. 12 is a chart showing the various scientific designations for brain activation in response to stimuli.

### DRAWING REFERENCE NUMERALS

10 Selection of Stimuli
11 Selection of Testing Equipment and Testing Environment
12 Selection of the Desired Skill Set
13 Finding High-Performing Individuals (HPIs) in the Desired Skill Set
14 Administering Stimulus and Brain Wave Test
15 Presenting raw test data to a computer Neural Network system to search for commonality and reduce noise.
16 Developing the Desired Skill Set group signature/template
21 Common Test and Environment
22 Selecting Special Skill Set #2
23 Finding High Performing Individuals in the Desired Skill Set
24 Administering Stimulus and Brain Wave Test
25 Presenting raw test data to a computer Neural Network system to search for commonality and reduce noise.
26 Developing the Desired Skill Set group signature/template
31 Common Test and Environment
32 Selecting Special Skill Set N
33 Finding High Performing Individuals in the Desired Skill Set N
34 Administering Stimulus and Brain Wave Test
35 Presenting raw test data to a computer Neural Network system to search for commonality and reduce noise.
36 Developing the Desired Skill Set group signature/template for Skill Set N
41 Common Test and Environment
42 Select Individual to be Tested
43 Administer Stimulus and Tests to Selected Individual
44 Data reduction and producing the Tested individual's brain template
45 Signature/Template Library
46 Search/Comparison Algorithm that compares the Tested Individual's template to the Special Sill Sets HPIs templates.
47 Determine if Tested Individual matches any Special Skill Groups templates.
101 Identification of High Performing Individuals of a particular skill set
102 Identification of Common Psychophysiological Characteristics
103 Identification of Stimuli for testing
104 Identification Physical Measures of Response to Stimuli
105 Test Stimuli on High Performing Individuals of the same skill set.
106 Determine confidence in match or no match
107 Administer Stimulus Set to test subjects
108 Correlate Candidate Responses
109 Refine Stimulus Set
110 Candidate Response Match?
111 Add to Group of Interest
112 Reject
201 Group of Interest Stimuli
202 Group of Interest. Signature
203 Stimulus Set Database
204 GOI Signature Database
205 Administer Stimulus Set to GOI Candidates
206 Correlate Candidate Response w/ GOI Response
207 Report Correlation
301 Electroencephalograph (EEG) data processing computer
302 EEG sensors
302a EEG sensor cable harness
303 Subject
306 Subject support structure
308 Subject input device
312 Visual display device
314 Control Computer
315 Data and Synchronization cable
316 System Support Structure
317 Stimuli Database
318 GOI Signature Database
501 EEG data processing computer
502 EEG sensor
503 Subject
504 EKG Sensor
505 Respiration Band
506 Chair
507 Shaker
508 Subject input device
509 EKG data processing computer
510 Audio output device
511 RF Transmitter/Receiver
512 Visual display device
513 Camera
514 Control computer
515 Data synchronization
516 System Support Structure
517 Stimuli Database
518 GOI Signature Database
710 Data bus
720 Display
730 User Input
740 Fixed Storage
750 Removable media
760 I/O controller
770 Memory
780 Processor
790 Network Interface
800 Network
810 Client
820 Client
830 Remote Platform
840 Server
850 Database
860 Data store
870 Data store
220

### DETAILED DESCRIPTION

According to an implementation of this disclosure, “Brain Activation Matching” techniques may be employed to match neurological and psychophysiological measures of individuals to other individuals or groups of individuals using data resulting from evoked and spontaneous brain activity. The distinctive qualities of neurological and psychophysiological measures of individuals or groups of individuals forms a brain activation signature for an individual or a group of individuals. Matching does not imply a perfect match of the psychophysiological response to stimuli but rather a similarity in gross or fine details of the response. Such similarity might be determined by analyzing features of brain activity or psychophysiological response, such as particular changes in brain activity in response to certain stimuli. Individual features or combinations of features may be characterized by their distance, correlation, covariance, or by other measure such as neural networks/artificial intelligence. An objective of Brain Activation Matching techniques is to identify individuals that match the brain activation response representative of a group-of-interest by exposing the individual and members of the Group-of-Interest (GOI) to stimuli eliciting brain activation. Individuals producing a brain activation response similar to the brain activation response representative of the group-of-interest will share neurological and cognitive processing characteristics with the group-of-interest. This similarity is useful for identifying promising candidates for inclusion with GOI.

A GOI composed of persons exceptionally skilled in a field of endeavor may be represented by a sub-set of high-performing individuals or experts from among the larger group. These selected high performers may be analyzed though psychophysiological testing using a suite of tests comprised of many stimuli in a consistent testing environment. Sensory stimuli types may include any number of images, sounds, or other sensory stimuli. Non-sensory stimuli include neurocognitive tasks, taskless intervals of brain monitoring or intervals of monitoring the brain in response to internally generated stimuli. Test results can be evaluated using well-known psychophysiological features and machine learning techniques employing deep neural networks and/or other techniques driven by artificial intelligence.

Measures of interest may include, but are not limited to, neurophysiological responses such as localized or global changes in measures of brain activity, directly or indirectly quantifiable, such as blood flow and related changes, blood oxygenation/hemoglobin concentration, changes in magnetic field or changes in light-absorption spectra, metabolic rates, or changes in the emission of energy by radioactive substances, changes in electromagnetic properties of single neurons or multitude of neurons, changes in ratios of chemical components or the production/release of chemical components by single cells or a multitude of cells. Additionally, measures of interest may include localized or global measures of morphology and physiology and changes thereof, measured by changes in brain structure including myelination, cortical thickness, structural connectivity, the central nervous system, and the peripheral nervous system. Other measures of interest include psychophysiological responses such as pupillometry, eye movement activity, direction-of-gaze activity, electrodermal activity, muscle activity, cardiography, heart rate activity, heart rate activity variability, blood pressure monitoring and respiration rate activity; psychological responses such as indicators of personality, learning styles, memory, executive function, response inhibition; psychophysical responses such as reaction time, signal detection; physiological changes such as hormone levels, blood glucose levels, oxygenation; and other markers distinguishing individuals and/or groups such as vocational skills.

Measurable responses can be elicited by different stimuli. Stimulus types include external stimuli, internal stimuli, sensory stimuli and neurocognitive task. External stimuli are those which are presented to a subject by an outside source such as an image or sound produced by an external device. Internal stimuli are those which originate from within the brain, mind, or body such as mental imagery of an object or imagining motor movements. Sensory stimuli are stimuli that directly activate sensory receptors. Sensory stimuli types include images, sounds, scents, tastes, vibrations, changes in temperature, pain, electrical stimulation, and/or chemical stimulation. Task stimuli elicit neurocognitive processes that represent cognitive function, psychophysical processes, and neurophysiological processes.

Stimulus datasets may be designed and populated to elicit responses from test subjects to focus on one or more neurocognitive characteristics of the groups and individuals. One application of the Brain Activation Matching system is to identify individuals that have brain activation responses similar to that of a population of a high-performers of a GOI but have not yet been trained to perform the tasks, duties or responsibilities of the GOI. In this case the stimulus datasets used to characterize the high-performing individuals of the GOI must, be populated with stimuli independent of information or skills learned during training for performance in the GOI. Non-learned stimuli are key to establishing the signature of the GOI in order to identify the characteristics of the group which underlie the success of High-Performing Individuals (HPIs) through training but unrelated to the training itself. Candidates for the GOI matching the brain activation response of HPIs of the GOI based on non-learned stimuli are likely to have the cognitive function, psychophysical processes, and neurophysiological processes similar to the high-performers and thus more likely to be successful in training for the duties, skills and responsibilities of the GOI.

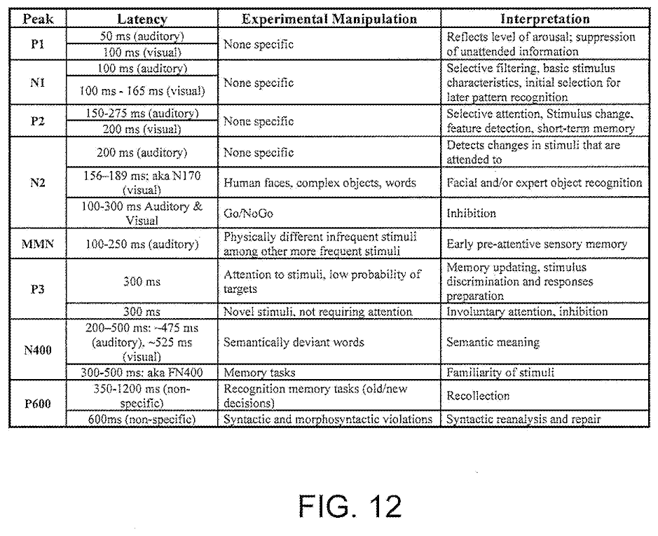

The table of FIG. 12 lists conventional electroencephalography (EEG) features in responses to various stimuli. For instance, the P-300 has proven very effective at indicating a test subject's level of recognition of sounds, words, numbers or images. Appropriate stimuli can be generated and presented to the test subject and responses recorded. Because the purpose of this system is to establish characteristic patterns of response to stimuli, the stimuli need not be limited to elucidating personality traits alone. Nor are the brain activation features limited to those of FIG. 12. Extraction, of signatures or templates by statistical methods, machine learning and artificial intelligence may identify distinguishing features or characteristics of brain activation response which are not named or previously characterized.

The response to stimuli may result in a set of measured values with fixed and known properties. One example is voltage resulting from brain activation measured by an electrode in contact with a particular location on the scalp and recorded by an EEG system. To classify a response, a set of these measured values in addition to a digital description of the particular stimulus can be input into a classifier such as a neural net (e.g., a multi-layer perceptron using backpropagation during training) or other classifier algorithm. The output may be a vector of values associated with stimulus that characterize a group composed of individuals who perform well, are experts in, or are talented in a particular field. This vector of outputs may be a refined version of the raw values measured and thus a good, general method of measuring the response to stimuli.

An example of how stimuli elicit neurological and psychophysiological responses indicative of personality traits is an electronic administration of a personality inventory, such as the Big Five Personality Test, which poses several statements to a test subject and asks the test subject to indicate how strongly the statement describes the test subject. For instance, the test may state that the test subject tends to find fault with others. In conventional paper and pencil test instruments, the test subject responds to the statement by marking one of five circles that represent degrees of agreement from “Strongly Disagree” to Strongly Agree.” An electronic administration of the same test can be accomplished by posing the same question to a test subject via text, visual representation or speech and monitoring the brain activation with EEG sensors. For instance, the N-400 (a distinctive negative voltage occurring about 400 ms post event) is an event-driven psychophysiological response triggered by stimuli that challenge the test subject with agreement or disagreement with self-concept. When instructed to assess how well statements describe the test subject, the amplitude of the N-400 may be proportional to the degree of agreement with the statement without the test subject explicitly respond. The preconscious brain response to the stimulus avoids self-reporting bias of the test subject which is often encountered in pencil and paper test instruments.

A signature for the high-performing members of the GOI based on non-learned stimuli facilitates association of individuals to that GOI not yet trained for the roles, functions and responsibilities of a GOI but potentially high-performers of the group and individuals partially or completely trained for the GOI.

In some applications of the Brain Activation Matching system, job performance may not be the most appropriate criteria for selecting the individuals most representative (the exemplars) of the GOI. For instance, social and service organizations may place greater value on community service or interpersonal skills rather than technical or organizational skills. A signature for the exemplar members of the GOI based on non-learned stimuli facilitates association of individuals not yet, familiar with the roles, functions and responsibilities of the GOI but potentially high performers of the group and individuals partially or completely familiar with the GOI.

In an implementation of this disclosure, responses to standardized test stimuli from multiple members of a highly specialized expert group may be compared using deep neural network techniques to identify the response characteristics common to the group and considered a signature of the group. Response signatures may be stored as a brain activation template for that expert group. A brain activation template is a digital representation of the unique features or characteristics of the brain activation response associated with an individual or group of individuals satisfying a common criteria for membership in that group. In a similar way, templates for various other specialized expert groups may be determined based on their response to the same standardized test stimuli. These templates may be compiled into a set of expert group templates. New test subjects may then be tested using the standardized test stimuli. The results of the new test subjects may be analyzed for matching with the set of expert group templates. Subjects producing a brain activation template with a strong similarity to a specific expert group brain activation template may be determined to have a high probability of performing well in the specialized area associated with the specific expert group associated with that specific template.

Additional benefits of Brain Activation Matching in accordance with an implementation of this disclosure, may be that a person not matching with one group could be guided to skill groups that he/she responds more similarly to the stimuli and therefore better suited for professional maturity through training and practice.

Implementations of this disclosure may facilitate the assessment of suitability for a particular job or task that spans the range from an individual applying for a single position open with a particular employer to many thousands of people trying to identify which of many positions they might be suited (e.g., military occupational specialty). Implementations of this disclosure can also be used for persons to explore vocations they are suited for, so that they can pursue appropriate fields of study to prepare them for entry or transition in the work force.

One problem with any new data stream can be knowing how to make sense of it, understand the information it contains, and exploit the information for some purpose. Extracting meaningful signals in data sequences recorded from psychophysiological response sensors may be accomplished by means of one or more data processing techniques and combinations of techniques that leverage conventional data features characterized by polarity, amplitude, latency and frequency; conventional signal processing techniques; statistical classifiers; machine learning techniques and artificial intelligence techniques.

For instance, EEG detects evidence of brain activity by electrical signals produced by neuronal activity that may be sensed by electrodes at the scalp of subject. Brain activity may be analyzed by characteristics of voltage changes observed by EEG. Brain activity may include weak signals having a significant quantity of noise. Implementations of the disclosure may identify correlations in brain activation data and extrapolate patterns based on well-known features of brain activity such as latency or amplitude of activation peaks, power in frequency bands, location of these features in relation to the brain and those determined by machine learning techniques employing deep neural networks and/or other artificial intelligence techniques. In an implementation of this disclosure, pattern matching may be employed to identify individuals whose brain responses to certain stimuli or at rest are similar to that of individuals who are very successful or talented in particular fields. As an example, if a young person's brain activity signature response to stimuli is similar in some way to an expert aircraft pilot, then it may be expected that the young person might also, with training, become an excellent pilot.

EEG observations may be quantified in conventional signal processing techniques in the time domain, frequency domain and the time-frequency domain. For example, a Fourier transformation of EEG data facilitates generation of numerical values of amplitude, phase, power, and frequency in order to generate meaningful values, ratios, percentages, graphically display arrays, trends; or set thresholds. Many quantitative EEG measures may be used to quantify slowing or attenuation of faster frequencies in the EEG. These include the calculation of power within different frequency bands (i.e., delta, theta, alpha, and beta); ratios or percentages of power in specific frequency bands; and spectral edge frequencies (based on the frequency under which x % of the EEG resides). These discrete values may then be compared between different regions, such as hemispheres, or between electrode-pair channels.

Conventional signal processing techniques include analyzing current, source density to estimate the location or origin of aspects of the EEG signal. Incorporating this information, filtered by the contribution of each channel to a measure of interest, the connectivity and composition of networks may be estimated and compared between different stimuli and test subjects. Such networks may also be identified using EEG signals acquired from the test subject at rest before or after stimulus presentation and may explain variance within subjects and between subjects.

Conventional signal processing techniques include time-compressed spectral arrays (“Spectrograms”) incorporate both power and frequency spectrum data and may be represented using color to show power at different frequencies as a function of time. Additional measures include amplitude integrated EEG, which continuously monitors brain activity by average ranges of peak-to-peak amplitudes displayed using a logarithmic scale, and the commercial Bispectral Index. Other nonparametric methods exist beyond Fourier transformation, including interval or period analysis and alternative transformation techniques. Parametric, mimetic, and spatiotemporal analyses are also available using a variety of computational methods and waveform analysis based on machine learning approaches trained on EEG recordings and other responses of interest. Basic measures of total power may be quantified and compared to performance characteristics to identify correlations that may be used to predict the recurrence of those performance characteristics. By way of example, a response pattern strongly associated with recognition is the “P-300” signal which has a characteristic positive deflection observed within a range of 250 to 600 milliseconds (ms) after being exposed to a recognized stimulus.

In some implementations of this disclosure, machine learning techniques may be based on statistical classification or computational neural networks. These machine learning techniques can enable the use of many different inputs without regard to a user's ignorance as to which inputs are important or even having a concept of what the inputs represent. In the case of a neural network such as a multi-layer perceptron, a large number of inputs can be used including those used to characterize the stimuli, the brain activity of the person being measured, and temporal delays used to model the brain's latency. As the network operates, weights on processing nodes may be adjusted nonlinearly using algorithmic feedback known as backpropagation. Over time and many empirical examples in a training data set, the input nodes that are unimportant to pattern classification can have their weights adjusted towards zero while those that are significant can have weights that increase. In this way, the neural network can “learn” (through weight adjustment) different patterns such as the brain wave patterns of exemplar humans who represent the best, most successful, and most talented individuals in particular domains (or as described above, expert groups). These different patterns can be expressed as a vector of the outputs of the neural network, but they can be quite recognizable and characteristic of the various exemplar humans. Thus, after training, the neural network can now classify persons as having brains that respond most similarly to one of the exemplars (or expert group template as discussed above). One obvious use for such a neural network may be to identify good fields of endeavor to suggest to young people. If a young person's brain wave response to certain stimuli is similar to an exemplar individual in a particular field, then it may be likely that the young person's brain is predisposed to enable success in that field.

The inputs to the machine learning techniques discussed herein can include the brain activity of a test subject who is responding to certain stimuli or brain activation at rest. Brain activity can vary by frequency and amplitude as well as the rates of change in frequency and amplitude. Furthermore, in addition to brain activity, other types of psychophysiological responses may be analyzed including but not limited to pupillary response, eye movement, heart rate, heart rate variability, respiration, and electrodermal activity. All of these factors can be inputs to the machine learning system because they are potentially correlated to brain response. For example, the frequency of measured brain activity at certain locations or globally can be correlated to state-of-mind, computational load on the brain, and certain personality characteristics such as the degree of extroversion/introversion.

A statistical classifier can be equivalent to a computational neural net for pattern recognition. Thus, implementations of this disclosure may employ statistical techniques in addition to neural networks, such as similar machine learning methods, or other artificial intelligence driven techniques.

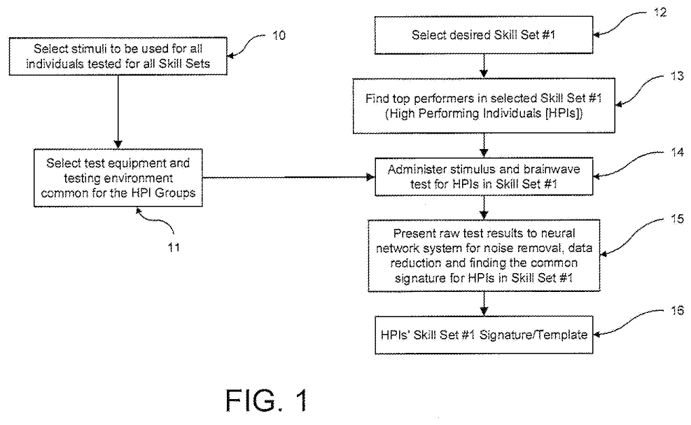

A computing device may execute various procedures for determining a brain activation signature or template for an expert group of High Performing Individuals (HPI), according to implementations of this disclosure. For example, FIG. 1 shows an example procedure, where stimuli 10 may be selected to be used as standardized stimuli for all individuals tested for all skill sets. This test may consist of a significant number of stimuli such as hundreds or thousands of images of various subjects, numbers, letters, objects, faces, abstract art, geometrical shapes, or 3-D presentations. An average human brain can process information in images faster than it can consciously perceive each image, corresponding with measurable brain activity. Taking into account the high processing speed for images, thousands of images may be presented in a short time. Images may be selected that have a bold subject and elicit a strong response.

In implementations of this disclosure, once the standardized stimuli selection has been made, the common test equipment and testing environment 11 can be selected. Since a test goal may be to measure variations between individuals, the test setup may be configured to reduce as many variables as possible.

Another benefit of implementations of this disclosure may be to determine if persons share brain activation or activation patterns/networks with HPI in response to external or internal stimuli or to tasks. The first step may be to select the desired skill set at 12 and then identify the HPI in this area at 13. For example, a first set of subjects may be composed of persons satisfying the first selection criteria of being high-performing individuals of a skill set. In some implementations a second set of subjects may be selected that satisfy a second selection criteria such as randomly selected from the general population, or a related baseline set of test subjects.

Referring again to FIG. 1, HPI identified at 13 may be presented with the standard stimuli at 14. For example, a sensory presentation device, such as a video screen or projection system may be communicatively connected to a computing device. The sensory presentation device may present the first sequence of stimuli from the standardized stimuli to the set of HPI. In some implementations, the sensory presentation device may also present the first sequence of stimuli to the second set of subjects.

For implementations in which EEG is used to sense and record e data sequence during or after presentation of the standardized stimuli, one or more electrodes connected to each of the HPI and in communication with the computing device may detect a first set of one or more voltage fluctuation sequences from each of the HPI. In some implementations, during or after presentation of the standardized stimuli, one or more electrodes operatively connected to each of the second set of subjects and in communication with the computing device may detect a second set of one or more voltage fluctuation sequences from each of the second set of subjects. Similarly, other types of psychophysiological responses such as pupillary response, eye movement, heart rate, heart rate variability, respiration, and electrodermal activity may be sensed and recorded separately or in combination with EEG.

Once the HPIs complete the test or as they complete the test, their raw test data can be submitted at 15 to a computing device implementing machine learning techniques that can look for commonality in brain activation data among the subjects of the expert group. For example, a neural network executing on the first computing device may determine a pattern of voltage fluctuations from EEG data that are characteristic of the HPI of desired Skill Set #1. This signature may be stored as a brain activation template and associated, with the HPI of desired Skill Set #1 at 16. In some implementations, the standard stimulus dataset 10, may be presented to a second set of test subjects populated with individuals from the general population to produce a second set of voltage fluctuations characteristic of the second set of test subjects. A neural network of 15 may determine a pattern of voltage fluctuations characteristic of the first set of test subjects and not characteristic of the second set of test subjects. This pattern of voltage fluctuations characteristic of the first set of test subjects and not characteristic of the second set of test subjects may be selected as the signature for the HPI and stored as a template at 16.

Because the raw psychophysiological datasets collected may be very large (e.g., gigabytes), it is advantageous to create a e of the psychophysiological data common to the top performers of the desired skill group and most distinctive of the group with respect to the general population or some other reference population. e can identify commonalities and differences among many large, complex and potentially noisy datasets to create the signatures. The discriminating qualities of the resulting signature encoded in the brain activation template is correlated with the number of top performers in the group representative of the desired skill group being evaluated. The more top performers included in the desired skill group, the more discriminating the signature and template.

Determining the brain activation signature for a GOI from multiple psychophysiological data sequences representative of the GOI may be accomplished by means of one or more data processing techniques and combinations of data processing techniques such as conventional signal processing, statistical methods, machine learning techniques, neural networks and artificial intelligence techniques.

Data collected for one or more additional psychophysiological measures of brain activity may be substituted for EEG voltage fluctuations. In some implementations, machine learning techniques such as neural networks may execute on computing devices such as one or more remote servers executing in a cloud computing environment in communication with a local computing device and/or sensory presentation device as discussed herein.

In some implementations, the procedure discussed with respect to FIG. 1 may include providing, by the first computing device, a recommendation for a selection of subjects from among a third set of test subjects based on the pattern of voltage fluctuations. For example, a third set of subjects may be presented with the standardized stimuli and their individual psychophysiological responses to the stimuli encoded as their brain activation template may be compared to the brain activation template associated with the HPI stored in the processing computer. Templates representing the psychophysiological responses of a subset of subjects within the third set of individuals may be determined to exhibit a similarity with the HPI template that exceeds a selection threshold value. In response to this determination, this subset of the third set of subjects forms a fourth set of individuals which may be recommended for consideration for training and/or performing the desired skill set associated with the HPI.

In some implementations, recommendations as discussed herein may be provided to other systems such as employee recruitment systems or components of enterprise human resources information systems and serve as a basis for further functionality of those systems. In some implementations, recommendations may be provided to an interface for a user of a computing device.

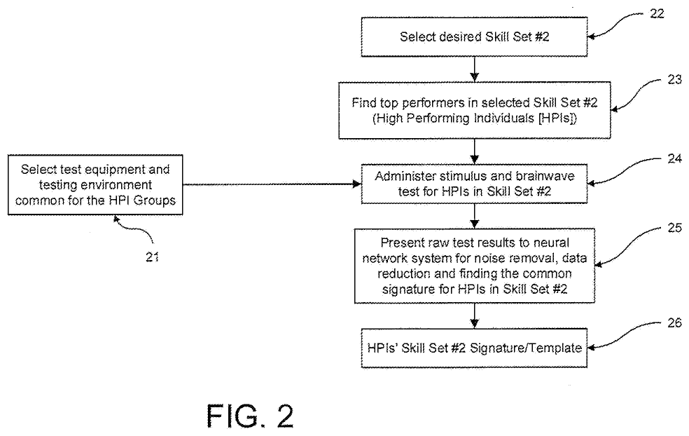

FIG. 2 shows a process similar to FIG. 1 for identifying a desired Skill Set #2, 22 then selecting the top performers of that Skill Set, 23. The same stimuli test and conditions of FIG. 1 are used in step 21 of FIG. 2 and may be administered at 24 to guide association of one or more test subjects to desired Skill Sets #1 or #2. Similar to the process of FIG. 1, neural network processing, 25 may be performed on the group of experts' raw test data to generate a signature and template of the group.

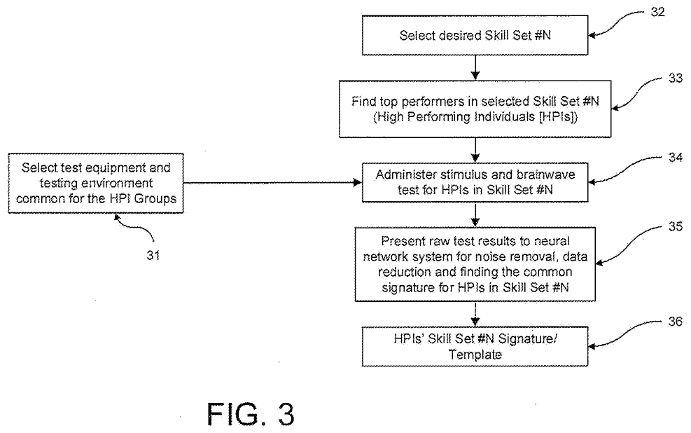

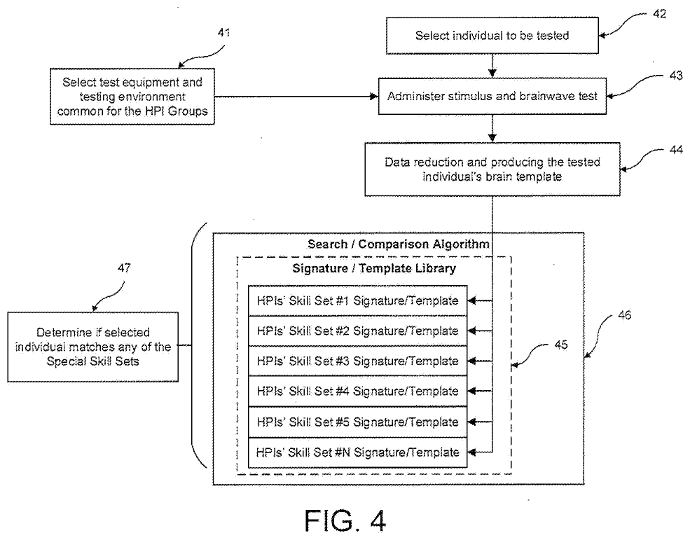

FIG. 3 expands FIG. 2 with basically the same process but selecting the Nth desired Skill Set, 32. By compiling a library of signatures/templates for a large number of desired Skill Sets, it will become possible to associate an individual with the Skill Set most closely matching his or her psychophysiological response to a stimulus dataset. FIG. 4 focuses on the processing steps to associate an individual, 42 with one or more signatures/templates of Skill Sets in a Signature/Template Library, 45 by Search/Comparison Algorithm, 46. The process of FIG. 4 is similar to FIG. 3 but instead of testing HPI to generate a signature/template for the desired skill group, the testing is administered to an individual of unknown skill set. The goal is to determine the degree of matching the individual to one or more of the desired Skill Set groups.

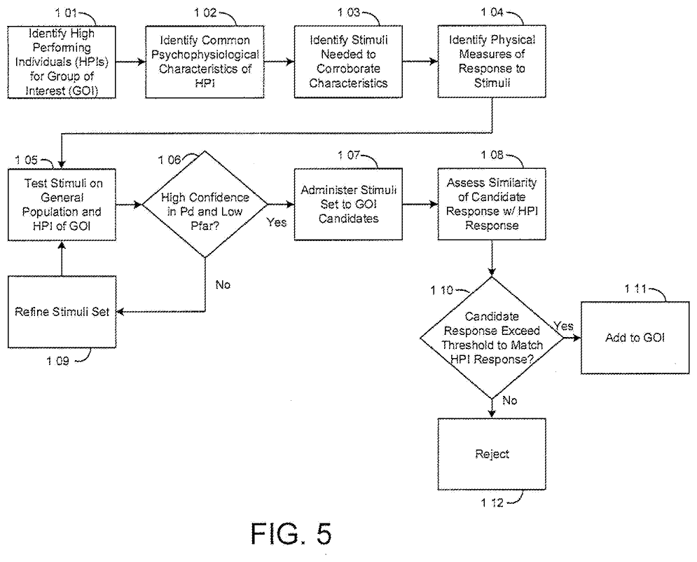

In FIG. 5, several steps are illustrated that enable the objective prediction of suitability of a candidate to a particular task or organizational function. The first step, 1 01, of the process may identify the HPI in a Group of Interest (GOI) that performs a particular task or organizational function. In step 1 02, the HPI can be evaluated to identify the neurological and psychological characteristics and traits common to the HPI of the GOI which enable them to excel in performance of duties and responsibilities of the GOI. In step 103, stimuli or tasks may be identified which will result in psychophysiologic responses which can be observed by the sensors of the system, additionally, responses during rest might be observed 1 04. Stimuli or tasks of step 1 03 and the physical measures of step 1 04 may be evaluated as effective predictive indicators of suitability for a particular task or function by testing people from the general population and HPI. The psychophysiological response of the HPI to the stimulus set is compared to that of the general population in step 1 06. One or more stimulus datasets which may include external and internal stimuli and tasks may be optimized for predictive performance of the signature associated with a particular GOI.

In step 1 06, the brain activation response common to the HPIs is compared to that of the general population. If the signature of the GOI is distinctly different from that of the general population so that the HPI are accurately detected as belonging to the GOI with a high degree of probability (Pd), low false positives and low false alarm rate (Pfar), then the set of stimuli may be validated to be reliably predictive of members of the GOI and, can be administered to candidates for the GOI. If not, then the stimulus set may be modified in step 1 09 and re-evaluated in steps 1 05 and 1 06 until the stimulus set is deemed sufficiently predictive.

Once the stimulus set is validated as predictive with high Pd and low Pfar, it can be administered to candidates for the GOI in step 107. The psychophysiological response of candidates to the stimulus set may be compared to that of the HPI response to the same stimulus set. Measures of similarity between the candidate response compared to the GOI signature predicts how well the candidate matches the response of the HPI and thus probability that the candidate could also be a strong performer in the GOI; step 1 10. If the strength of match exceeds a threshold value, the candidate may be deemed to be a fit in the GOI, step 1 11. If not, the candidate may be deemed unlikely to perform well in the GOI.

Measures of similarity or matching between psychophysiological response data of an individual to one or more brain activity signatures of GOIs, may be accomplished by means of data processing techniques including conventional signal processing, statistical methods, machine learning techniques, clustering, neural networks, artificial intelligence techniques and combinations thereof. Psychophysiological response data may be comprised of the psychophysiological response data sequences or templates derived from the response data sequences.

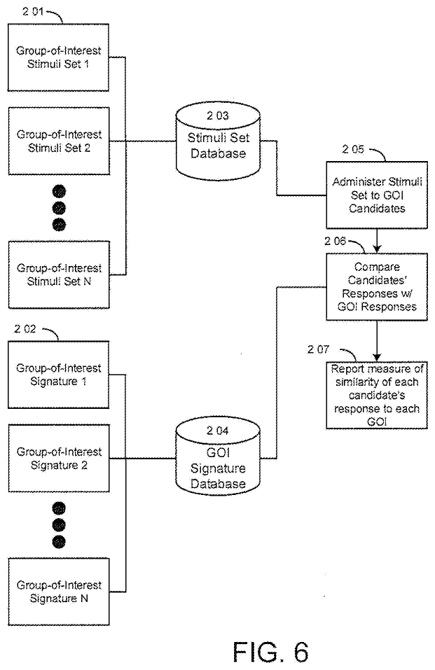

FIG. 6 illustrates an extension of the process and system described in FIG. 5 in which one or more candidates are evaluated for a single Group-of-Interest (GOI). The process in FIG. 6 evaluates the degree of fit of candidates to multiple GOIs. The steps of FIG. 6 may be implemented for multiple tasks or functions so that a library of diagnostic stimulus sets 2 01 is populated in database 2 03. The distinctive signatures for HPI of each task 2 02 may populate a signature database, 2 04. The library of stimulus data sets may be administered to candidates for the corresponding GOIs in step 2 05. The response of the candidate to the stimuli may be compared to those of the GOI signatures in step 2 06. Measures of similarity between the candidates' response to stimuli and the GOI signatures may be tabulated in a report 2 07.

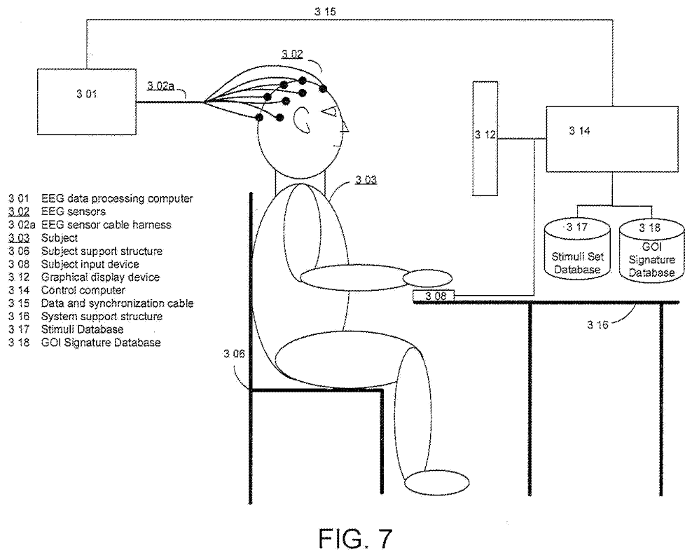

FIG. 7 illustrates an embodiment of this invention. A test subject 3 03 is seated before a visual display device. 3 12. In this particular embodiment, stimuli 3 17 may be visual in nature and are displayed on the visual display device. 3 12. Stimulus elements, for instance still images, may be sequentially displayed at intervals and durations programed by the method of rapid serial visual presentation (RSVP) which is well known in the art. Visual presentation of stimuli by RSVP typically displays images at a rate of 5 to 10 images per second depending.

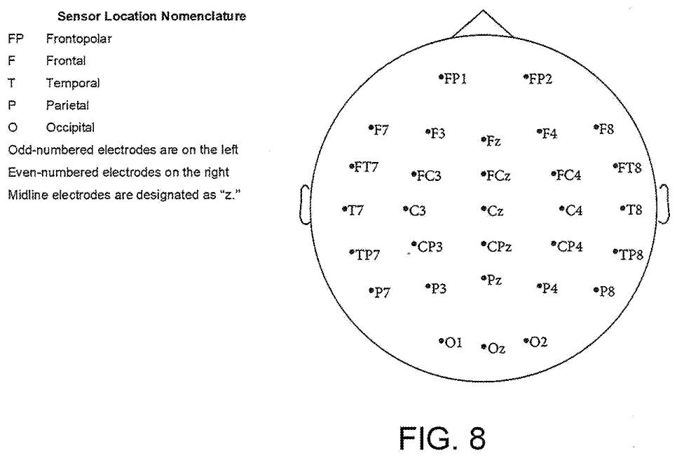

One or more sensors 3 02 may be arranged on the test subject's scalp according to locations illustrated in FIG. 8 for a 30-channel system, in an embodiment. The number and location of sensors may differ depending upon the EEG system employed or stimulus types presented to the subject 3 03. The sensors, data collection and processing collectively facilitate EEG. Sensor locations may be selected to obtain strong signals for specific brain activity resulting from the RSVP stimuli. Brain activity may have characteristic properties such as shape, polarity and latency which is well established in the art. The table of FIG. 12 presents well known brain activation features and some of their properties.

In some implementations of this disclosure, communication means 3 15 may provide a channel for data to be transferred between EEG data processing computer 3 01 and control computer 3 14. Channel 3 15 may also provide the timing data needed for EEG data processing computer to know when stimuli is presented to the subject 3 03 so that latency of brain activation can be computed. This channel may be a wired or wireless connection, and may use any data format or protocol known in the art.

Test subject input device 3 08 may be used to keep the subject 3 03 attentive to the visual display device 3 12 while presentation of the stimulus data is in progress. For instance, the subject may be asked to press a button on a keyboard or activating a switch when a particular image or type of image is displayed. Input device 3 08 may also be used to measure test subject response time, motion inhibition response and similar psychophysiological responses. Likewise, input device 3 08 may be used in conjunction with other stimulus presentation types or tasks.

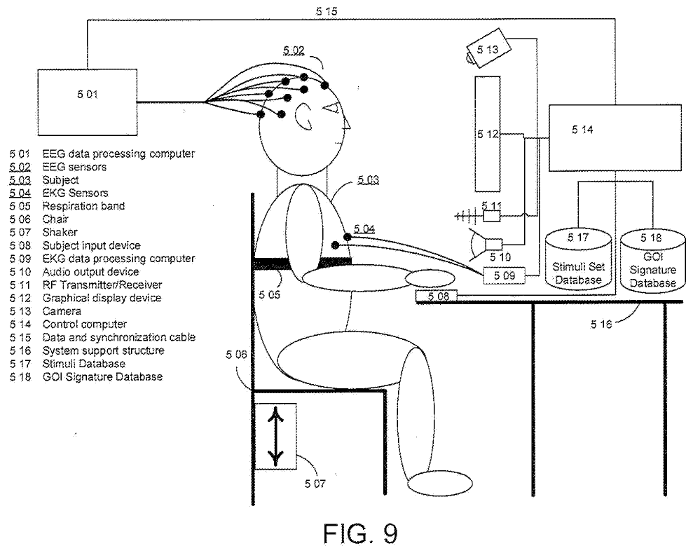

FIG. 9 illustrates a system in which stimuli may be delivered to multiple human senses and multiple psychophysiological response sensors of several types are employed to observe and record psychophysiological response data sequences to the multi-modal stimulation for each psychophysiological response sensor. Stimulus generating components may include the audio output device 5 10 and shaker 5 07 which is capable of imparting signals affecting the sense of touch of the subject. For clarity in the figure, stimulus generators affecting the senses of other sensory receptors such as taste, smell or temperature are not shown but could form a part of this system.

Other stimulation methods might be employed to stimulate various sensory neurons evoking corresponding sensations. Sensations may include mechanical (e.g. vibration, pressure), nociceptive, temperature, itch, auditory, gustatory, proprioceptive, olfactory components or any combination thereof. A specific sensation might be evoked by different methods, e.g. the sensation of “itch” might be caused by electrical stimulation or the application of chemicals, the sensation of “hot” might be caused by the application of a hot element to the body or electrical or chemical stimulation and so forth.

Psychophysiological sensor types of the system may be selected from among a group including those described in FIG. 9 and may include the EEG system components 5 01 and 5 02; electrocardiogram (EKG) sensors 5 04 and EKG data processing computer 5 09; respiration band 5 05, RF transmitter/receiver 5 11, which can be used to measure heart rate, heart rate variability and respiration using RF Doppler vibrometry and electrodermal activity; and a camera to observe pupillary response, eye movement and muscle tension. In alternative embodiments, different subsets of these sensors may be used. The system configured in this way can produce one or more stimuli and observe one or more psychophysiological response data sequences to the stimuli.

Description of a preferred embodiment; Visual stimuli, EEG for Single GOI

In a preferred embodiment of the invention, the process of FIG. 5 and the instrumentation of FIG. 7 may be employed to assess the fit of candidates for a single GOI.

### Operation of Preferred Embodiment

In an embodiment, of the invention, the process of FIG. 5 may be employed to establish the characteristic response signature of a subset of a particular GOI assessed to be HPIs of that GOI. Through iteration of testing and refinement, a set of visual stimuli may be validated to distinguish between known members of the GOI and known non-members of the GOI with a high probability of detection and low false alarm rate. The validated stimulus set can then be administered to individuals and resulting psychophysiological response data sequences observed by EEG elements 3 01 and 3 02 of FIG. 7 to determine if individuals fit the signature of the GOI or not. Elements of the stimulus set may be reordered within a stimulus set presentation or mixed amongst the various stimulus sets presented.

### Embodiment 2; Single Non-Visual Input, EEG Sensors and Single GOI

In an alternative embodiment of the invention, the process of FIG. 5 and the instrumentation of FIG. 9 may be employed to assess the fit of candidates for a single GOI using stimulus sets evoking psychophysiological response data sequences using non-visual stimuli.

### Operation of Embodiment 2

In this embodiment of the invention, the process of FIG. 5 may be employed to establish the characteristic response signature of a subset of a particular GOI assessed to be HPI of that GOI. Through iteration of testing and refinement, a set of non-visual stimuli may be validated to distinguish between known members of the GOI and known non-members of the GOI with a high probability of detection and low false alarm rate. The validated stimulus set may be composed of non-visual external stimuli and is administered to individuals by RSXP where X can comprise any non-visual stimulus modality. Presentation frequency may vary with the presented stimulus to account for physiological processes, readying or resetting the sensory receptor to be receptive for the next stimulus. The resulting psychophysiological response data sequences may be produced by EEG elements 3 01 and 3 02 of FIG. 9 which are used to determine if the response signature of individuals fits the characteristic of the GOI or not. The elements of the stimulus set may be reordered within a stimulus set presentation or mixed amongst the various stimulus sets presented.

### Embodiment 3; Visual Stimuli, EEG and Multiple GOI

An alternative embodiment of the invention may be configured to assess the fit of one or more candidates to more than one GOI by RSVP or tasks and EEG.

### Operation of Embodiment 3

In this configuration of the invention, the process of FIG. 5 may be employed to establish the characteristic signature response of HPI for each of more than one GOIs. For each of more than one GOIs, a set of visual stimuli may be validated to distinguish between known members of each GOI and known non-members of each GOI with a high probability of detection and low false alarm rate. The multiple stimulus sets associated with each GOI can then be administered to individuals by RSVP and resulting psychophysiological response data sequences produced by EEG elements 3 01 and 3 02 of FIG. 7 which are used to determine how well individuals fit each of the GOIs. The elements of the stimulus set may be reordered within a stimulus set presentation or mixed amongst the various stimulus sets presented.

### Embodiment 4; Non-Visual Stimuli, EEG and Multiple GOI

An alternative embodiment of the invention may be configured to assess the fit of one or more candidates to more than one GOI by RSXP and EEG.

### Operation of Embodiment 4

In this configuration of the invention, the process of FIG. 5 may be employed to establish the characteristic signature response of HPIs for each of more than one GOIs. For each of more than one GOIs, a set of non-visual stimuli may be validated to distinguish between known members of each GOI and known non-members of each GOI with a high probability of detection and low false alarm rate. The stimulus sets associated with each GOI can then be administered to individuals by RSXP and resulting psychophysiological response data sequences produced by EEG elements 3 01 and 3 02 of FIG. 9 which are used to determine how well individuals fit each of the GOIs. The elements of the stimulus set may be reordered within a stimulus set presentation or mixed amongst the various stimulus sets presented.

### Embodiment 5; RSVP, Non-EEG Sensors, Single GOI

An alternative embodiment of the invention may use RSVP to characterize a single GOI by observations of psychophysiological responses other than brain activity measured by EEG.

### Operation of Embodiment 5

In this embodiment of the invention, the process of FIG. 5 may be employed to establish the characteristic signature response of a subset of a GOI assessed to be HPI of that GOI. Through iteration of testing and refinement, a set of visual stimuli may be validated to distinguish between known members of the GOI and, known non-members of the GOI with a high probability of detection and low false alarm, rate. The validated stimulus set can then be administered to individuals by RSVP. The resulting psychophysiological response may be observed by instruments other than EEG sensors. Candidate sensors may include one or more cameras sensitive to the visible and non-visible components of the spectrum (e.g., infrared) to monitor pupillary response, eye movement, vasodilation, muscle tension, etc.; electrocardiogram for heart rate and heart rate variability; respiration band for respiration rate and abnormalities; RF Doppler vibrometry to observe heart rate, heart rate variability, respiration and muscle movements; skin resistivity measures electrodermal activity. Laser Doppler vibrometry may perform the same function as RF Doppler Vibrometry. There are many other sensor types for observing psychophysiological responses that are well known in the field of polygraphy that could also be employed measurements commonly used in this embodiment.

### Embodiment 6; RSVP, Non-EEG Sensors, Multiple GOIs

An alternative embodiment of the invention may use RSVP to characterize multiple GOIs by observations of psychophysiological responses other than brain activity measured by EEG.

### Embodiment 7; RSXP, Non-EEG Sensors. Single GOI

An alternative embodiment of the invention may use RSXP to characterize a single GOI by observations of psychophysiological responses other than brain activity measured by EEG.

### Embodiment 8; RSXP, Non-EEG Sensors, Multiple GOIs

An alternative embodiment of the invention may use RSXP to characterize multiple GOIs by observations of psychophysiological responses other than brain activity measured by EEG.

### Embodiment 9; RSVP and RSXP, EEG Sensors, Single GOI

An alternative embodiment of the invention may use a combination of RSVP and RSXP in conjunction with brain activity as measured by EEG to characterize a single GOI.

### Embodiment 10; RSVP and RSXP, EEG Sensors, Multiple GOI

An alternative embodiment of the invention may use a combination of RSVP and RSXP in conjunction with brain activity as measured by EEG to characterize multiple GOIs.

### Embodiment 11; RSVP and RSXP, EEG and Non-EEG Sensors, Single GOI

An alternative embodiment of the invention may use a combination of RSVP and RSXP in conjunction with brain activity to characterize a single GOI.

### Embodiment 12; RSVP and RSXP, EEG and Non-EEG Sensors, Multiple GOIs

An alternative embodiment of the invention may use a combination of RSVP and RSXP in conjunction with brain activity to characterize multiple GOIs.

### Embodiment 13; Multiple Candidates Evaluated Concurrently for Each of the Embodiments Above

### Embodiment 14; Brain Activation by Tasks Alone

An alternative embodiment of the invention may use one or more tasks to characterize one or more GOIs in conjunction with non-EEG observations.

### Embodiment 15; Brain Activation by Tasks and Stimulus

An alternative embodiment of the invention may use one or more tasks and stimulus set to characterize one ore more GOIs which may employ a combination of RSVP and RSXP and other presentation modes in conjunction with EEG and non-EEG observations.

### Embodiment 16; Resting State

An alternative embodiment of the invention may use resting state EEG to characterize one or more GOIs in conjunction with non-EEG data.

### Embodiment 17; Resting State and Stimulus

An alternative embodiment of the invention may use a combination of resting state EEG and a stimulus set to characterize one or more GOIs in conjunction with non-EEG data.

### Embodiment 18; in an Alternative Embodiment, Brain Activation

Matching may be configure to provide vocational guidance for people entering the workforce or people in the workforce striving to realize their full potential. For example, a new recruit entering military service makes a decision on branch of service or which occupational specialty the recruit may wish to pursue (e.g., Infantry, Armor, Logistics, mechanic, etc.). It would be informative to the recruit and the receiving service to have the recruit categorized as sharing brain activation or activation pattern/networks in response to stimuli (e.g., external or internal) or tasks with HPI one or more special skill sets within the service. The recruit, and service organization would then have significantly important information to assist in decisions for investing time, energy and money training for the soldier.

In Embodiment 19, the brain activation signatures can be kept on file when a soldier enters the military. Soldiers sometimes face physical and emotional trauma resulting in illnesses such as Traumatic Brain Injury (BTI), Post-Traumatic Stress Disorder (PTSD) or depression. After exposure to potential traumatic events the soldier could be evaluated with the same stimulus data sets used at entrance to the military and results compared to his or her original brain activation recording to assess changes in stimulus response potentially indicating the presence and severity of injury.

In an alternative embodiment of the invention, multiple candidates may be evaluated simultaneously or asynchronously during a fixed interval of time. Each candidate may be subjected to the same stimulus sets which may be presented in the same or different order.

### Embodiment 20; Dynamic Selection of Stimulus Sets

Candidates can be evaluated by dynamically selected stimulus datasets which are automatically selected by the system based on how well a candidate matches GOIs at high levels of abstraction. For instance, if a candidate's responses match better with a GOI for general engineering compared to other vocational types, the system may select stimulus sets from a lower tier of engineering disciplines that provide more specificity in engineering such as mechanical, electrical or software. The number of levels of specificity for any particular functional category may not be limited.

### Embodiment 21; Matching Individuals to Multiple GOIs

Over time as the Brain Activation Matching invention builds a library of signatures/templates for numerous GOI, these groups can be assembled to allow a test subject to be matched to a GOI. This embodiment could assemble thousands of GOI signatures/templates to enable very specific matching.

### Embodiment 22; Synthetic GOIs

This embodiment enables creation of signatures/templates for GOIs not previously identified. For example, test subjects exposed to a common stimulus dataset that do not match well to any GOI in the signature/template library may be grouped/paired to form their own GOIs based on shared psychophysiological response traits. The resulting synthetic GOIs could be then examined to identify the skills, interests, and abilities common to the individuals comprising the synthetic GOIs.

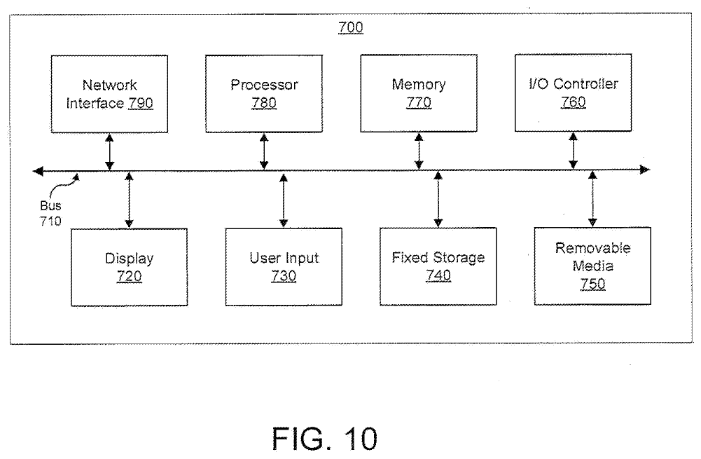

Implementations of the present disclosure may be implemented in and used with a variety of component and network architectures. FIG. 10 illustrates an example computing device 700, such as a computer, suitable for implementations of the present disclosure. The computing device 700 may include a bus 710 which interconnects major components of the computing device 700, such as a central processor 780; a memory 770 (typically RAM, but which may also include ROM, flash RAM, or the like); an input/output controller 760; a user display 720, such as a display screen via a display adapter; a user input interface 730, which may include one or more controllers and associated user input devices such as a keyboard, mouse, and the like, and may be closely coupled to the I/O controller 760; fixed storage 740, such as a hard drive, flash storage, Fibre Channel network, SAN device, SCSI device, and the like; and a removable media component 750 operative to control and receive an optical disk, flash drive, and the like.

The bus 710 may allow data communication, between the central processor 780 and the memory 770, which may include read-only memory (ROM) or flash memory (neither shown), and random access memory (RAM) (not shown), as previously noted. The RAM may generally be the main memory into which the operating system and application programs are loaded. The ROM or flash memory can contain, among other code, the Basic Input-Output system (BIOS) which controls basic hardware operation such as the interaction with peripheral components. Applications resident with the computing device 700 may generally be stored on and accessed via a computing device readable medium, such as a hard disk drive (e.g., fixed storage 740), an optical drive, floppy disk, or other storage medium.

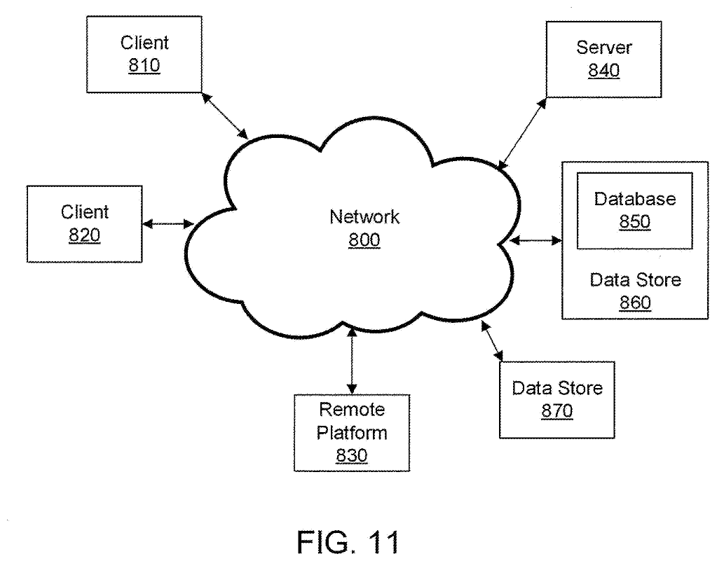

The fixed storage 730 may be integral with the computing device 700 or may be separate and accessed through other interfaces. A network interface 790 may provide a direct connection to a remote server via a telephone link, to the Internet via an internet service provider (ISP), or a direct connection to a remote server via a direct network link to the Internet via a POP (point of presence) or other technique. The network interface 790 may provide such connection using wireless techniques, including digital cellular telephone connection, Cellular Digital Packet Data (CDPD) connection, digital satellite data connection or the like. For example, the network interface 790 may allow the computing device to communicate with other computing devices via one or more local, wide-area, or other networks, as shown in FIG. 11.

Many other devices or components such as those of FIGS. 7 and 9 may be connected in a similar manner. Conversely, all of the components shown in FIG. 10 need not be present to practice the present disclosure. The components can be interconnected in different ways from that shown. The operation of a computing device such as that shown in FIG. 10 is readily known in the art and is not discussed in detail in this application. Code to implement the present disclosure can be stored in computing device-readable storage media such as one or more of the memory 770, fixed storage 740, removable media 750, or on a remote storage location.

FIG. 11 illustrates an example network arrangement according to an implementation of the disclosure. One or more clients (e.g., 810, 820), consisting of local computing devices, smart phones, tablet computing devices, and the like may connect to other devices via one or more networks 800. The network 800 may be a local network, wide-area network, the Internet, or any other suitable communication network or networks, and, may be implemented, on any suitable platform including wired and/or wireless networks. The clients may communicate with one or more servers 840 and/or databases 850. The devices may be directly accessible by the clients 810, 820, or one or more other devices which may provide intermediary access to devices such as a server 840 which provides access to resources stored in a database 850. The clients 810, 820 also may access remote platforms 830 or services provided by remote platforms 830 such as cloud computing arrangements and services. The remote platform 830 may include one or more servers 840 and/or databases 850.

The architecture of FIG. 10 enables an embodiment of the Brain Activation Matching system in which operational components are distributed and in communication with each other via a Network 800 as illustrated in FIG. 11. Network 800 may provide communication between components in close proximity such as a laboratory setting. Alternatively, components may be distributed over great distances. Data Stores, 860 and 870 may be accessed by multiple Servers 820 or Clients 810 and 820. In its simplest configuration; a Server may consist of a Processor 780 but could also be populated with additional components such as Network interface 790, Memory 770, I/O Controller 760. Removable Media 750, and Fixed Storage 740. Simple configurations of Client nodes 810 and 820 may consist of components such as EEG sensors, 5 02, EEG Data Processing Computer, 5 01 Display 720 and 5 12, and User Input device 730 and 5 08. Client nodes may include other stimulus presentation devices such as the audio output device 510 and shaker 5 07 and psychophysiological sensor types such as camera 5 13, RF transmitter 511, EKG sensors, 5 04, and respiration band 5 05. All components necessary to practice the Brain Activation Matching system reside in one or more of the clients, servers or data stores. In this embodiment, stimulus datasets may be exchanged between local or remote components of hardware and software via interfaces with the Network, 800. Stimulus datasets may be reproduced and presented to the test subject at either local or remote locations, including taste, smell and touch.

More generally, various implementations of the present disclosure may include or be implemented in the form of computing device-implemented processes and apparatuses for practicing those processes. Implementations also may be implemented in the form of a computing device program product having computing device program code containing instructions implemented in non-transitory and/or tangible media, such as floppy diskettes, CD-ROMs, hard drives, USB (universal serial bus) drives, or any other machine readable storage medium, wherein, when the computing device program code is loaded into and executed by a computing device, the computing device becomes an apparatus for practicing implementations of the disclosure. Implementations also may be implemented in the form of computing device program code, for example, whether stored in a storage medium, loaded into and/or executed by a computing device, or transmitted over some transmission medium, such as over electrical wiring or cabling, through fiber optics, or via electromagnetic radiation, wherein when the computing device program code is loaded into and executed by a computing device, the computing device becomes an apparatus for practicing implementations of the disclosure. When implemented on a general-purpose microprocessor, the computing device program code segments may configure the microprocessor to create specific logic circuits. In some configurations, a set of computing device-readable instructions stored on a computing device-readable storage medium may be implemented by a general-purpose processor, which may transform the general-purpose processor or a device containing the general-purpose processor into a special-purpose device configured to implement or carry out the instructions. Implementations may be enabled using hardware that may include a processor, such as a general-purpose microprocessor and/or an Application Specific Integrated Circuit (ASIC) that implements all or part of the techniques according to implementations of the disclosure in hardware and/or firmware. The processor may be coupled to memory, such as RAM, ROM, flash memory, a hard disk or any other device capable of storing electronic information. The memory may store instructions adapted to be executed by the processor to perform the techniques according to implementations of the disclosure.

The foregoing description, for purpose of explanation, has been described with reference to specific implementations. However, the illustrative discussions above are not intended to be exhaustive or to limit implementations of the disclosure to the precise forms disclosed. Many modifications and variations are possible in view of the above teachings. The implementations were chosen and described in order to explain the principles of implementations of the disclosure and their practical applications, to thereby enable others skilled in the art to utilize those implementations as well as various implementations with various modifications as may be suited to the particular use contemplated.

1. A system comprising:
a. a psychophysiological response sensor subsystem comprised of at least one of a psychophysiological response sensor selected from at least one of a psychophysiological sensor types and combinations thereof to observe and record at least one of a psychophysiological response measure resulting from evoked and spontaneous brain activity;
b. a computing device comprising a processor in communication with said psychophysiological response sensor subsystem;
c. a non-transitory, computer-readable medium in communication with said processor and storing instructions that, when executed by said processor, cause said processor to perform operations comprising:
d. a sequence of stimuli comprised of at least one of a stimulus element of at least one of a stimulus type:
e. a set of sequences of stimuli:
f. presenting by a sensory presentation device a first sequence of stimuli to: (i) a first set of subjects associated with a first selection criteria and (ii) a second set of subjects associated with a second selection criteria;
g. detecting a first set of psychophysiological response data sequences, said first set of psychophysiological response data sequences comprising a sequence of psychophysiological response measures of interest from each of said first set of subjects;
h. detecting a second set of psychophysiological response data sequences, said second set of psychophysiological response data sequences comprising a sequence of psychophysiological response measures of interest from each of said second set of subjects;
i. determining, by a first data processing means, a brain activation signature for said first set of subjects comprised of a pattern of psychophysiological response data sequences characteristic of said first set of psychophysiological response data sequences and not characteristic of the second set of psychophysiological response data sequences; and
j. extracting a brain activation template comprising a digital representation of the distinctive characteristics of said brain activation signature for said first set of subjects and store in the computing device.

2. The system of claim 1 further comprising:
a. presenting, by said sensory presentation device, the first sequence of stimuli to a third set of subjects comprised of at least one subject associated with a third selection criteria;
b. detecting psychophysiological response data sequences for each of the third set of subjects;
c. creating a brain activation template for each of the third set of subjects;
d. computing measures of similarity by a second data processing means operating on said brain activation template of each of the third set of subjects and said brain activation template representative of said first set of subjects;
e. select from among the third set of subjects a fourth set of subjects for which the similarity of the templates of the third group exceeds a first predefined threshold criterion of similarity with respect to the first set of subjects and a fifth set of subjects whose templates fail to exceeds said first threshold criterion of similarity with respect to the first set of subjects.

3. The system of claim 1 wherein:
a. said first set of subjects is representative of a first group-of-interest and said second set of subjects is a general population;
b. selecting from said set of sequences of stimuli a first of at least one of a selected sequence of stimuli selected from at least one of a group of standardized sequences of stimuli and sequences of stimuli optimized for each of N groups-of-interest to result in a unique signature for each of N groups-of-interest;
c. presenting by a sensory presentation device one or more of said selected sequence of stimuli to: (i) each of N groups-of-interest each with a unique selection criteria and (ii) said general population;
d. determining, by said first data processing means, a brain activation signature for each of said N groups of interest; and
e. extract a brain activation template for each of said N groups of interest and store in a signature database of said computing device.

4. The system of claim 3, further comprising a recommendation for potential association of candidate individuals with the N groups-of-interest based on the pattern of psychophysiological response wherein:
a. presenting, by said sensory presentation device, said selected sequence of stimuli to a third set of subjects comprised of at, least, one subject associated with a third selection criteria;
b. detecting psychophysiological response data sequences for each of the third set of subjects;
c. creating a brain activation template for each of the third set of subjects;
d. computing measures of similarity by said second data processing means operating on said brain activation template for each of said third set of subjects and said brain activation templates for each of said N groups-of-interest;
e. tabulating the measure of similarity for each of said third set of subjects for each of said N groups-of-interest and stored in said computing device;
f. recommending each of said third set of subjects for association with each of said N groups-of-interest for which each of said third set of subjects exceeds a second predefined threshold criterion of similarity for each of said N groups-of-interest;
g. creating a fourth set of subjects from the third set of subjects that fail to exceed said second predefined threshold criterion of similarity for any of the N groups-of-interest.

5. The system of claim 4 further comprising:
a. computing measures of similarity by said second data processing means for brain activation templates for each of said fourth set of subjects compared to the brain activation response templates all the other subjects of said fourth set of subjects, tabulating the results and storing in said computing device;
b. creating one or more subgroups of similar response of said fourth set of subjects that share measures of similarity exceeding a third predefined threshold criterion of similarity;
c. extracting a brain activation template for each of said subgroups of similar response representative of each of said subgroups of similar response and storing in said computing device as a template for a synthetic group-of-interest.

6. The system of claim 4 wherein:
a. selecting for each of said third set of subjects a second and subsequent sequences of stimuli from among said set of sequences of stimuli based on the measure of similarity tabulated for each of said third set of subjects for each of said N groups-of-interest stored in said computing device;

7. A system comprising:
a. a psychophysiological response sensor subsystem comprised of at least one of a psychophysiological response sensor selected from at least one of a psychophysiological sensor types and combinations thereof to observe and record at least one of a psychophysiological response measure resulting from evoked and spontaneous brain activity:
b. a computing device comprising a processor in communication with said psychophysiological response sensor subsystem;
c. a non-transitory, computer-readable medium in communication with said processor and storing instructions that, when executed by said processor, cause said processor to perform operations comprising:
d. a sequence of stimuli comprised of at least one of a stimulus element of at least one of a stimulus type:
e. a set of sequences of stimuli:
f. presenting by a sensory presentation device a first sequence of stimuli to: (i) a first subject associated with a first selection criteria and (ii) a second subject associated with a second selection criteria;
g. detecting a first set of psychophysiological response data sequences, said first set of psychophysiological response data sequences comprising a sequence of psychophysiological response measures of interest from said first subject;
h. detecting a second set of psychophysiological response data sequences, said second set of psychophysiological response data sequences comprising a sequence of psychophysiological response measures of interest from said second subject;
i. creating a first brain activation template for said first subject and a second brain activation template for said second subject;
j. computing measures of similarity by a second data processing means operating on said first brain activation template and said second brain activation template;
k. indicating that said first brain activation template and said second brain activation template are matched when said measure of similarity exceeds a fourth predefined threshold criterion of similarity.

8. The System of claim 7 wherein:
a. Said first selection criteria and second selection criteria are the same.
